(19) United States
(12) Patent Application Publication
(10) Pub. No.: US 2023/0307109 A1
(43) Pub. Date: Sep. 28, 2023

(54) INTERACTION BETWEEN PHARMACEUTICAL DISPENSING DEVICES

(71) Applicant: Tech Pharmacy Services, LLC, Fort Lee, NJ (US)

(72) Inventors: Omer EINAV, Kfar-Monash (IL); Doron SHABANOV, Tzur-Yigal (IL); Tamir BEN DAVID, Tel-Aviv (IL); Anthony Joseph SPERO, Queensbury, NY (US); Eyal LIVSCHITZ, Givat Shmuel (IL); Thomas A. MCKINNEY, Boonton, NJ (US); Moshe LIBERMAN, Yehud (IL)

(73) Assignee: Tech Pharmacy Services, LLC, Fort Lee, NJ (US)

(21) Appl. No.: 18/015,087

(22) PCT Filed: Jul. 7, 2021
(86) PCT No.: PCT/IB2021/056079

§ 371 Date: Jan. 9, 2023

Related U.S. Application Data
(60) Provisional application No. 63/049,147, filed on Jul. 8, 2020.

(51) Int. Cl. G16H 20/13; G16H 40/20; G16H 40/40

(52) U.S. Cl. CPC G16H 20/13; G16H 40/20; G16H 40/40

(57) ABSTRACT

The present invention relates to methods of distributing pharmaceuticals in a pharmaceutical dispensing system comprising two or more pharmaceutical dispensers configured to store, package and dispense pharmaceuticals in batches according to a schedule and/or immediately dispense non-scheduled pharmaceuticals in response to at least one non-scheduled demand, where some of the pharmaceutical dispensers comprise a limited pharmaceutical storage capacity. The method comprises distributing pharmaceuticals scheduled to be dispensed in batches to one pharmaceutical dispenser and distributing only a subset of non-scheduled pharmaceuticals to another pharmaceutical dispenser. The determination of the subset is performed by (a) scoring each non-scheduled pharmaceutical by a degree of disruption caused to said schedule by dispensing thereof by the first pharmaceutical dispenser; and (b) finding a subset of said non-scheduled pharmaceuticals which minimizes said disruption based on said scoring and also fits in said limited pharmaceutical storage capacity of the second pharmaceutical dispenser.

### RELATED APPLICATION/S

This application claims the benefit of priority of U.S. Provisional Pat. Application No. 63/049,147 filed on 8 Jul. 2020, the contents of which are incorporated herein by reference in their entirety.

### FIELD AND BACKGROUND

The present invention, in some embodiments thereof, relates to interaction between pharmaceutical dispensing devices and, more particularly, but not exclusively, to active optimization of interactions between pharmaceutical dispensing devices.

Additional background art includes U.S. Pat. No. US5907493A discloses “a pharmaceutical dispensing system including a plurality of standardized or universal-type cells disposed in a large array called a module. One or more modules may be disposed in a given pharmacy, and one or more pharmacies may be monitored by a central computer”.

U.S. Pat. No. US7860724B2 discloses “a system and method for optimized management of pharmacy workflow, specifically, workflow associated with fulfillment of prescription orders for medications and health-related products in a pharmacy environment. A computer-controlled system coordinates and controls pharmacy workflow to sequence prescriptions for fulfillment in a most efficient path thereby minimizing a cost function associated with fulfillment of the prescription order. The system coordinates and controls prescription order fulfillment from automated and non-automated storage locations and can be easily adapted to the specific layout and level of automation desired by the operator”.

U.S. Pat. Application No. US20040210341A1 discloses “systems and methods for the remote dispensing of packaged and non-packaged medical products using networked communications systems. A preferred embodiment of the invention utilizes a network to provide for the secure delivery of confidential patient information and the sending of dispense instructions to a remote dispensing station. A preferred embodiment of the present invention relates to systems and methods of dispensing samples of drugs or other medical products. Another preferred embodiment of the invention provides a system and method for dispensing non-prescription medications”.

U.S. Pat. Application No. US20100030667A1 discloses “methods and systems adaptive storage and management of pharmaceutical product containers at a pharmacy are described. Pharmaceutical product containers are managed so that the containers for more-frequently-used pharmaceutical products are stored among plural storage locations more-efficiently accessible to a pharmacy workstation. Containers for less-frequently-used pharmaceutical products are managed so that the containers for such products are stored among the storage locations which are less-accessible to the pharmacy workstation. As the frequency of pharmaceutical product usage changes, the inventory of pharmaceutical product containers is managed adaptively so that the containers used most frequently are stored at locations more-easily accessible to the pharmacy workstation, thereby facilitating fulfillment of prescriptions by pharmacy personnel. Further efficiencies may be achieved through use of an optical positioning system providing directed placing and picking of pharmaceutical product containers. The methods and system have particular application to management of partially-full pharmaceutical product containers which may be repeatedly taken from and returned to inventory”.

U.S. Pat. Application No. US20050043011A1 discloses “a system coupled to one or more vending machines, such as soft drink vending machines, via a wireless data link can acquire operational vending data, for example sales data, hardware status, and product temperature. The system can compile data from multiple vending machines dispersed across a geographic area such as a city or state. The system can include software that refines the vending operations of one or more such vending machines based on analysis of acquired data, taking into consideration other information such as market, business, seasonal, or environmental factors. Refining vending operations can include adjusting product offerings, relocating vending machines, replicating favorable conditions, and addressing unexpected sales variations”.

### SUMMARY

Following is a non-exclusive list including some examples of embodiments of the invention. The invention also includes embodiments which include fewer than all the features in an example and embodiments using features from multiple examples, also if not expressly listed below.

Example 1. A method of distributing pharmaceuticals in a pharmaceutical dispensing system, said pharmaceutical dispensing system comprising at least one first pharmaceutical dispensing machine configured to store, package and dispense pharmaceuticals in batches according to a pharmaceutical dispensing schedule, at least one second pharmaceutical dispensing machine configured to store, package and dispense non-scheduled pharmaceuticals in response to at least one non-scheduled demand, said at least one second pharmaceutical dispensing machine comprising a limited pharmaceutical storage capacity which is less than a pharmaceutical storage capacity of said at least one first pharmaceutical dispensing machine, the method comprising:
distributing pharmaceuticals to be dispensed according to said pharmaceutical dispensing schedule to said at least one first pharmaceutical dispensing machine and distributing only a subset of non-scheduled pharmaceuticals to said at least one second pharmaceutical dispensing machine;
said distributing only a subset of non-scheduled pharmaceuticals to said at least one second pharmaceutical dispensing machine comprising determining said subset by:
   (a) scoring each non-scheduled pharmaceutical by a degree of expected disruption caused to said pharmaceutical dispensing schedule by dispensing thereof by said at least one first pharmaceutical dispensing machine;
   (b) finding a subset of said non-scheduled pharmaceuticals which reduces said expected disruption based on said scoring and also fits in said limited pharmaceutical storage capacity of said at least one second pharmaceutical dispensing machine.

Example 2. The method according to example 1, wherein said determining said subset of non-scheduled pharmaceuticals further comprises scoring said each non-scheduled pharmaceutical by a degree of expected disturbance caused to at least one user.

Example 3. The method according to example 1 or 2, wherein said at least one second pharmaceutical dispensing machine being closer to said at least one user than said at least one first pharmaceutical dispensing machine.

Example 4. The method according to any one of examples 1-3, wherein pharmaceuticals that are expected to be dispensed together are distributed to the same pharmaceutical dispensing machine albeit of their scoring.

Example 5. The method according to any one of examples 1-4, wherein said determining said subset of non-scheduled pharmaceuticals further comprises scoring each non-scheduled pharmaceutical by a degree of potential urgency of dispensing of non-scheduled pharmaceuticals.

Example 6. The method according to any one of examples 1-5, wherein said determining said subset of non-scheduled pharmaceuticals further comprises scoring each non-scheduled pharmaceutical by frequency of dispensing of non-scheduled pharmaceuticals.

Example 7. The method according to any one of examples 1-6, wherein said determining said subset of non-scheduled pharmaceuticals further comprises scoring each non-scheduled pharmaceutical by specific subpopulations of users, their proximity to said at least one second pharmaceutical dispensing machine and the past history data of dispensing of non-scheduled pharmaceuticals to said specific subpopulation.

Example 8. The method according to any one of examples 1-7, wherein said determining said subset of non-scheduled pharmaceuticals further comprises scoring each non-scheduled pharmaceutical by an anticipation of needs of non-scheduled pharmaceuticals.

Example 9. The method according to any one of examples 1-8, wherein said anticipation of needs are based on data from one or more of run models, real-time data, historical data, physical location of users expected to need the pharmaceuticals, expected location of users at an expected time of dispensing.

Example 10. The method according to any one of examples 1-9, wherein said determining said subset of non-scheduled pharmaceuticals further comprises scoring each non-scheduled pharmaceutical by an anticipation of problems in the pharmaceutical dispensing system.

Example 11. The method according to any one of examples 1-10, wherein said anticipation of problems are based on data from one or more of scheduled maintenance, run models and historical data.

Example 12. The method according to any one of examples 1-11, wherein said at least one first pharmaceutical dispensing machine is further configured to store, package and dispense non-scheduled pharmaceuticals in response to at least one non-scheduled demand.

Example 13. The method according to any one of examples 1-12, wherein said at least one second pharmaceutical dispensing machine is further configured to store, package and dispense pharmaceuticals in batches according to a pharmaceutical dispensing schedule.

Example 14. The method according to any one of examples 1-13, wherein said dispense non-scheduled pharmaceuticals in response to at least one non-scheduled demand is done immediately after the at least one non-scheduled demand is received.

Example 15. The method according to any one of examples 1-14, wherein said expected disruption caused to said schedule is caused by disruption of a pharmaceutical dispensing process of said pharmaceuticals in batches.

Example 16. The method according to any one of examples 1-14, wherein said expected disruption of said pharmaceutical dispensing process of said pharmaceuticals in batches is caused by said dispensing of said non-scheduled pharmaceuticals in response to said at least one non-scheduled demand.

Example 17. The method according to any one of examples 1-16, further comprising dispensing said pharmaceuticals according to said schedule.

Example 18. A pharmaceutical dispensing system, comprising:
a. at least one first pharmaceutical dispensing machine for storing, packing and dispensing one or more pharmaceuticals in batches according to a pharmaceutical dispensing schedule;
b. at least one second pharmaceutical dispensing machine for storing, packing and dispensing one or more non-scheduled pharmaceuticals in response to at least one non-scheduled demand;
wherein said one or more non-scheduled pharmaceuticals included in said at least one second pharmaceutical dispensing machine reduce potential disruption in said pharmaceutical dispensing schedule in comparison to including said one or more non-scheduled pharmaceuticals in said at least one first pharmaceutical dispensing machine.

Example 19. The pharmaceutical dispensing system according to example 18, wherein said one or more non-scheduled pharmaceuticals are chosen according to a scoring of each non-scheduled pharmaceutical by a degree of expected disturbance to at least one user, said chosen one or more non-scheduled pharmaceuticals being those that reduces said expected disturbance.

Example 20. The pharmaceutical dispensing system according to examples 18 or 19, wherein said at least one second pharmaceutical dispensing machine being closer to said at least one user than said at least one first pharmaceutical dispensing machine.

Example 21. The pharmaceutical dispensing system according to examples 18-20, wherein pharmaceuticals that are expected to be dispensed together are distributed to the same pharmaceutical dispensing machine albeit of their scoring.

Example 22. The pharmaceutical dispensing system according to examples 18-21, wherein said one or more non-scheduled pharmaceuticals are chosen according to a scoring of each non-scheduled pharmaceutical by a degree of potential urgency of dispensing of non-scheduled pharmaceuticals, said chosen one or more non-scheduled pharmaceuticals being those higher degree of potential urgency.

Example 23. The pharmaceutical dispensing system according to examples 18-22, wherein said one or more non-scheduled pharmaceuticals are chosen according to a scoring of each non-scheduled pharmaceutical by a degree of frequency of dispensing of non-scheduled pharmaceuticals, said chosen one or more non-scheduled pharmaceuticals being those higher degree of frequency.

Example 24. The pharmaceutical dispensing system according to examples 18-23, wherein said one or more non-scheduled pharmaceuticals are chosen according to a scoring of each non-scheduled pharmaceutical by specific subpopulations of users, their proximity to said second pharmaceutical dispensing machine and the frequency of dispensing of said non-scheduled pharmaceuticals to said specific subpopulation, said chosen one or more non-scheduled pharmaceuticals being those dispensed by said specific subpopulations of users, which are the closest to said second pharmaceutical dispensing machine and having a higher degree of frequency.

Example 25. The pharmaceutical dispensing system according to examples 18-24, wherein said one or more non-scheduled pharmaceuticals are chosen according to a scoring of each non-scheduled pharmaceutical by a degree of anticipation of needs of non-scheduled pharmaceuticals, said chosen one or more non-scheduled pharmaceuticals being those with higher degree of anticipation.

Example 26. The pharmaceutical dispensing system according to examples 18-25, wherein said anticipation of needs are based on data from one or more of run models, real-time data, historical data, physical location of users expected to need the pharmaceuticals, expected location of users at an expected time of dispensing.

Example 27. The pharmaceutical dispensing system according to examples 18-26, further comprising scoring each non-scheduled pharmaceutical by an anticipation of problems in the pharmaceutical dispensing system.

Example 28. The pharmaceutical dispensing system according to examples 18-27, wherein said anticipation of problems are based on data from one or more of scheduled maintenance, run models and historical data.

Example 29. The pharmaceutical dispensing system according to examples 18-28, wherein said at least one first pharmaceutical dispensing machine is further configured for storing, packing and dispensing non-scheduled pharmaceuticals in response to at least one non-scheduled demand.

Example 30. The pharmaceutical dispensing system according to examples 18-29, wherein said at least one second pharmaceutical dispensing machine is further configured for storing, packing and dispensing pharmaceuticals in batches according to a pharmaceutical dispensing schedule.

Example 31. The pharmaceutical dispensing system according to examples 18-30, wherein said dispense non-scheduled pharmaceuticals in response to at least one non-scheduled demand is done immediately after the at least one non-scheduled demand is received.

Example 32. The pharmaceutical dispensing system according to examples 18-31, wherein said expected disruption caused to said schedule is caused by disruption of a pharmaceutical dispensing process of said pharmaceuticals in batches.

Example 33. The pharmaceutical dispensing system according to examples 18-32, wherein said expected disruption of said pharmaceutical dispensing process of said pharmaceuticals in batches is caused by said dispensing of said non-scheduled pharmaceuticals in response to said at least one non-scheduled demand.

Example 34. A pharmaceutical dispensing system, comprising:
a. a first pharmaceutical dispensing machine for storing, packing and dispensing one or more pharmaceuticals in batches according to a pharmaceutical dispensing schedule and also for storing, packing and dispensing one or more non-scheduled pharmaceuticals in response to at least one non-scheduled demand;
b. a second pharmaceutical dispensing machine for storing, packing and dispensing one or more pharmaceuticals in batches according to a pharmaceutical dispensing schedule and also for storing, packing and dispensing one or more non-scheduled pharmaceuticals in response to at least one non-scheduled demand;
wherein said one or more pharmaceuticals destined to be dispensed in batches and said one or more non-scheduled pharmaceuticals are chosen according to a known population of users located in proximity to said at least one first and said at least one second pharmaceutical dispensing machines.

Example 35. A pharmaceutical dispensing system located in a same building comprising a plurality of departments, comprising:
a. a first pharmaceutical dispensing machine configured for storing, packing and dispensing one or more pharmaceuticals;
b. a second pharmaceutical dispensing machine configured for storing, packing and dispensing one or more pharmaceuticals;
wherein said one or more pharmaceuticals are chosen according to a known population of users located in proximity to said at least one first and said at least one second pharmaceutical dispensing machines.

Example 36. A pharmaceutical dispensing machine located in proximity to users, for storing, packing and dispensing one or more pharmaceuticals; said one or more pharmaceuticals are chosen according to the requirements of said users located in proximity to said pharmaceutical dispensing machine.

Example 37. A method of choosing which pharmaceuticals to add into a pharmaceutical dispensing machine, comprising:
a. collecting data regarding pharmaceutical dispensing needs of users in proximity to said pharmaceutical dispensing machine;
b. adding pharmaceuticals found in point (a) into said pharmaceutical dispensing machine.

Example 38. A method of distributing pharmaceuticals in a pharmaceutical dispensing system, said pharmaceutical dispensing system comprising at least one first pharmaceutical dispensing machine for storing, packing and dispensing pharmaceuticals according to a pharmaceutical dispensing schedule, at least one second pharmaceutical dispensing machine for storing, packing and dispensing non-scheduled pharmaceuticals in response to at least one non-scheduled demand, the method comprising:
a. distributing pharmaceuticals to be dispensed according to said pharmaceutical dispensing schedule to said at least one first pharmaceutical dispensing machine;
b. distributing said non-scheduled pharmaceuticals to said at least one second pharmaceutical dispensing machine;
wherein said non-scheduled pharmaceuticals are chosen according to the expected needs of users located in proximity to said at least one second pharmaceutical dispensing machine.

Example 39. A method of distributing pharmaceuticals in a pharmaceutical dispensing system, said pharmaceutical dispensing system comprising at least one first pharmaceutical dispensing machine configured to store, package and dispense pharmaceuticals and located in proximity to at least one first population of users, at least one second pharmaceutical dispensing machine configured to store, package and dispense pharmaceuticals and located in proximity to at least one second population of users, the method comprising:
a. distributing pharmaceuticals to said at least one first pharmaceutical dispensing machine according to the expected needs of said at least one first population of users;
b. distributing pharmaceuticals to said at least one second pharmaceutical dispensing machine according to the expected needs of said at least one second population of users.

Example 40. A method of distributing pharmaceuticals in a pharmaceutical dispensing system, said pharmaceutical dispensing system comprising at least one first pharmaceutical dispensing machine configured to store, package and dispense pharmaceuticals according to a pharmaceutical dispensing schedule, at least one second pharmaceutical dispensing machine configured to store, package and dispense non-scheduled pharmaceuticals in response to at least one non-scheduled demand and also configured to provide pharmaceuticals according to a scheduled demand, the method comprising:
a. distributing pharmaceuticals to be dispensed according to said pharmaceutical dispensing schedule to said at least one first pharmaceutical dispensing machine;
b. distributing said non-scheduled pharmaceuticals to said at least one second pharmaceutical dispensing machine, such that said second machine does not include any of said pharmaceuticals dispensed according to said scheduled demand;
wherein said non-scheduled pharmaceuticals are chosen according to the expected needs of users located in proximity to said at least one second pharmaceutical dispensing machine.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, microcode, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a schematic pharmaceutical dispensing system 100 employed in a facility, according to some embodiments of the claimed invention;

FIG. 2 is a schematic representation of an exemplary interaction between pharmaceutical dispensing machines and the pharmaceutical dispensing system, according to some embodiments of the invention;

FIG. 3 is a flowchart of an exemplary method of organization of pharmaceuticals between one or more pharmaceutical dispensing devices, according to some embodiments of the invention; and

FIG. 4 is a flowchart of an exemplary method of interaction between pharmaceutical dispensing machines and a pharmaceutical dispensing system, according to some embodiments of the invention.

### DESCRIPTION OF SPECIFIC EMBODIMENTS OF THE INVENTION

The present invention, in some embodiments thereof, relates to interaction between pharmaceutical dispensing devices and, more particularly, but not exclusively, to active optimization of interactions between pharmaceutical dispensing devices.

An aspect of some embodiments of the invention relates to optimization of distribution of pharmaceuticals between pharmaceutical dispensing devices. In some embodiments, the distribution of pharmaceuticals is performed using correlation parameters of pharmaceutical dispensing requirements in a computational logistics system. In some embodiments, the pharmaceutical dispensing requirements that help in the decision of the allocation of pharmaceuticals in a specific machine use one or more of the following correlation parameters: allocating pharmaceuticals in proximity to patients, allocating specific pharmaceuticals in proximity to a relevant subset of patients that require said specific pharmaceuticals, allocating pharmaceuticals in anticipation of needs, allocating pharmaceuticals in anticipation of problems, allocating pharmaceuticals according to frequency of requests, allocating pharmaceuticals according to type of pharmaceutical, allocating pharmaceuticals in view of known users, allocating pharmaceutical according to real-time and/or historical prescription information, allocating ready-to-be-dispensed envelopes between pharmaceutical dispensing machines, allocating pharmaceuticals according to the expected location of the user, allocating pharmaceuticals according to pharmaceutical dispensing schedules, allocating pharmaceuticals according to a level of disturbance and/or disruption that will cause dispensing from a certain pharmaceutical dispensing machine, where the disturbance is to one or more of a user and a pharmaceutical dispensing schedule. In some embodiments, the distribution of pharmaceuticals is optimized according to the needs of the pharmaceutical dispensing system. In some embodiments, the distribution of pharmaceuticals is optimized according to the needs of the users/patients. In some embodiments, the distribution of pharmaceuticals is optimized according to the needs of the dedicated personnel (physician, nurses, etc.).

An aspect of some embodiments of the invention relates to optimization of interactions between pharmaceutical dispensing devices in a same location. In some embodiments, the interactions enable optimization of distribution of workload between pharmaceutical dispensing devices. In some embodiments, all pharmaceutical dispensing devices are in communication with a server, which coordinates the activity of each pharmaceutical dispensing device according to the needs of the facility. In some embodiments, a pharmaceutical dispensing device provides pharmaceuticals to a different zone in the same facility, for example in case of local device dysfunction, to provide support for other local device in a different zone. In some embodiments, the location of each pharmaceutical dispensing device is based on optimizing the minimal distance from users, patients and/or dedicated personnel.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Pharmaceutical Dispensing System

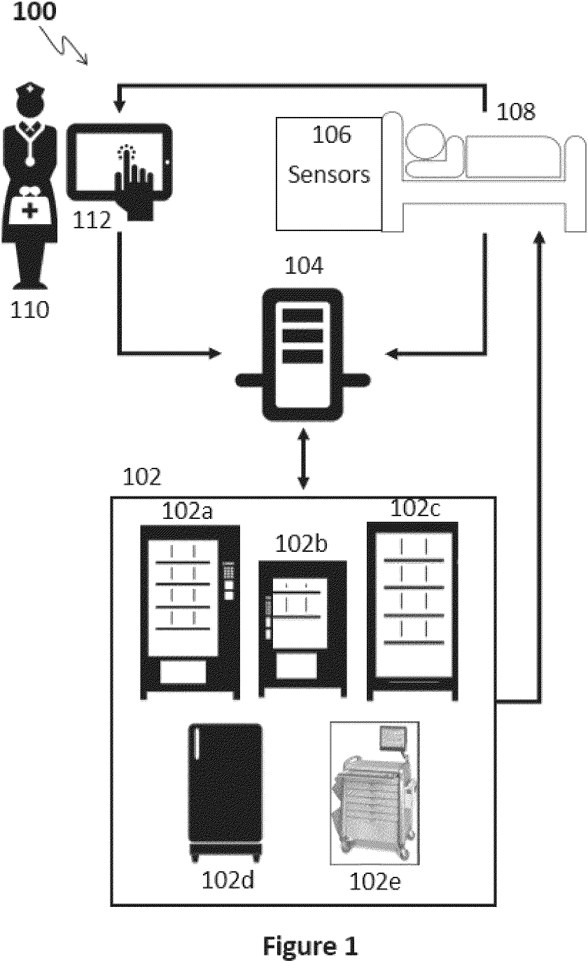

Referring now to the drawings, FIG. 1 illustrates a flowchart of a schematic pharmaceutical dispensing system 100 employed in a facility, according to some embodiments of the claimed invention. In some embodiments, the pharmaceutical dispensing system comprises a plurality of pharmaceutical dispensing machines 102 placed in different locations in the facility. In some embodiments, one or more devices are placed in the same location. In some embodiments, a combination of devices are placed in the same location. In some embodiments, the devices are capable of storing and dispensing pharmaceuticals. In some embodiments, the devices only storage pharmaceuticals. In some embodiments, the plurality of pharmaceutical dispensing machines include, for example, the following devices:
A large pharmaceutical dispensing machine 102a, which is adapted to store, package and dispense a large quantity of pharmaceuticals;
A small pharmaceutical dispensing machine 102b, which comprises the same capabilities of the large machine 102a but with fewer pharmaceuticals in it;
A bulk storage cabinet 102c, which comprises a variety of pharmaceuticals and medical materials (e.g. liquid drugs, IV, bandages, insulin, pill storage) in bulk. In some embodiments, the small and/or the large pharmaceutical dispensing machine packages pharmaceuticals as the circumstance arises (also known as PRN - “pro re nata”) and/or first dose pharmaceuticals for a single patient. In some embodiments, adapted and authorized personnel can access the bulk storage cabinet 103 when necessary.

A refrigerator 102d, which comprises a variety of pharmaceuticals and/or medical materials that require special storage temperatures. In some embodiments, adapted and authorized personnel can access the refrigerator 102d when necessary.

A nursing cart 102e, which are optionally locked and opened by adapted and authorized personnel. In some embodiments, authorized personnel can be a nurse, a physician or any other authorized personnel.

In some embodiments, as mentioned above, each pharmaceutical dispensing device comprises a certain storage capacity. In some embodiments, the storage capacity depends on one or more of the number of pharmaceutical containers in the device, the size of the pharmaceutical containers, the size of the pharmaceuticals that will dictate the number of pharmaceuticals that can be storage in a pharmaceutical container and the type of pharmaceutical (pills, liquid, and powder).

In some embodiments, pharmaceutical dispensing devices can be equipped with dedicated units configured to store and dispense pharmaceutical that require special temperature storage conditions (cold) and/or need to be dispensed in a special form (liquid or powder). In some embodiments, the dedicated units will take the place of some pharmaceutical containers in the pharmaceutical dispensing machine. In some embodiments, dedicated units will take more place in the pharmaceutical dispensing machine in comparison to the regular pharmaceutical containers.

In some embodiments, the location of any of the abovementioned pharmaceutical dispensing devices is chosen either arbitrarily and/or taking under consideration the distance that a user/patient and/or personnel needs to do in order to reach the pharmaceutical dispensing devices. In some embodiments, the distance is less than 300 meters, optionally less than 200 meters, optionally less than 100 meters.

In some embodiments, any of the above pharmaceutical dispensing machines may include one or more of prescription pharmaceuticals, over the counter (OTC) pharmaceuticals and items that are ‘other than pharmaceuticals’ - for example first aid materials like plasters in a variety of different sizes and shapes, disinfectants, bandages, alcohol, tourniquets, small, medium and large sterile gauze dressings, sterile eye dressings, triangular bandages, crêpe rolled bandages, safety pins, disposable sterile gloves, disposable sterile masks, tweezers, scissors, alcohol-free cleansing wipes, sticky tape, thermometer, skin rash cream, such as hydrocortisone or calendula, cream or spray to relieve insect bites and stings, antiseptic cream, distilled water for cleaning wounds, eye wash and eye bath, pocket mask for CPR, dental kit, sterile knifes/bistoury, syringes and more.

As used herein, the term “dedicated personnel” and/or “physician” and/or “doctor” and/or “nurse” may refer to an individual that has acquired, either through specialized education, experience, and/or training, a level of expertise in regards to the subject activity. A dedicated personnel may be qualified to provide assessment data documenting the subject physiological status and provide an assessment to aspects or domains of the subject physiological status that require expert-level judgement. A dedicated personnel may be a doctor, a nurse and/or a pharmacist. A dedicated personnel may be known to the subject or may be completely anonymous.

In some embodiments, any of the above dispensing machines and/or storage units assists in the pharmaceutical dispensing service. In some embodiments, different devices of the system are used in combination to provide the pharmaceuticals in the pharmaceutical dispensing service. In some embodiments, each of the abovementioned devices is monitored and controlled by the system. In some embodiments, any of the above dispensing machines and/or storage units comprise a plurality of pharmaceuticals according to their physical capacities, for example, certain dispensing machine may comprise 30 different pharmaceuticals, while other may comprise 100 or 300 or any quantity of different pharmaceuticals.

In some embodiments, the plurality of pharmaceutical dispensing machines 102 are connected to at least one server 104. In some embodiments, the server comprises all the information regarding users, patients, personnel, pharmaceuticals, insurance, budget, costs, timetables for pharmaceutical deliveries from suppliers; also past, present and future information regarding pharmaceutical needs with relation to patients and the cases related to those needs (see below - Exemplary Pharmaceutical Dispensing System Server).

In some embodiments, the server 104 includes an assessment tool software.

In some embodiments, a plurality of sensors and measuring devices 106 are connected or are in physical communication with the patient 108 and are adapted to measure and collect a plurality of data regarding the physiological, physical and mental state of the patient 108 at any moment. In some embodiments, the measurements collected by the plurality of sensors are sent to the server 104 in order to be analyzed by the assessment tool software.

In some embodiments, dedicated personnel 110 that monitor the patient, can access the pharmaceutical dispensing system using dedicated hardware/software 112 using a dedicated graphical user interface (GUI). In some embodiments, the GUI can be a tablet, a smartphone and/or a computer. In some embodiments, the GUI is a device located near the bed of the patient. In some embodiments, the pharmaceutical dispensing system can be accessed remotely using any kind of electronic device using cloud-based technology. In some embodiments, the dedicated personnel can use the GUI to inquire regarding one or more of: the current location (e.g. not in room, doing physiotherapy), the expected location (e.g. will be at physiotherapy at 10am), the usual location (e.g. 4^{th} floor, room 406, bed 6), of a specific patient and perform changes in the pharmaceutical dispensing process according to the information.

### Exemplary Storing, Packaging and Dispensing of Pharmaceutical Dispensing Machines

In some embodiments, the pharmaceutical dispensing machine comprises one or more modules. In some embodiments, optionally, the pharmaceutical dispensing machine comprises one or more backup and/or redundant hardware and/or module adapted to perform the same and/or similar and/or different functions. In some embodiments, optionally, said at least one module and said at least one backup and/or redundant hardware and/or module perform tasks at the same time. In some embodiments, the pharmaceutical dispensing machine comprises one or more modules selected from the group consisting of: a pharmaceutical array module, a pharmaceutical tote module, a pharmaceutical operational modules section and a mechanical arm module. In some embodiments, the modules comprise one or more sensors that monitor the functioning and/or the performance and/or the demands of the modules. In some embodiments, the one or more sensors are activated remotely by a user via a dedicated server.

In some embodiments, the modules in the pharmaceutical dispensing machine do not work independently to each other. In some embodiments, the modules work as a series of actions performed by each module. For example, the control module receives information about the next batch of pharmaceuticals to be dispensed. The mechanical arm module is activated to reach for a specific pharmaceutical located in the pharmaceutical array module. In some embodiments, the pill is engaged by a pill engagement mechanism and the mechanical arm module transports the pharmaceutical to an envelope taken from the envelope module. In some embodiments, single pills are engaged each time, meaning the mechanical arm module picks a single pill and transport it to its destination (e.g. an envelope). Once all the necessary pharmaceuticals are inserted in the envelope, the envelope is then closed, for example, by a crimper module, and a printer module marks the envelope with the relevant information. Then the mechanical arm module transports the envelope to the pharmaceutical tote module, where eventually a user will pick up the tray with all the envelopes and/or the specific envelope. Exemplary description of this technology can be found in US application 16/379,835, US patent 10,614,916, PCT application IB2020/052052, PCT application IB2020/053082, PCT application IB2020/053080 and PCT application IB2020/055232, which are herein incorporated in their entirety by reference into the specification.

### Example of Preparation of a Batch of Pharmaceuticals

In a scenario of a hospital having several rooms in a floor, in each room several patients, it scheduled to deliver the morning medicines at 08:00 in the morning. The pharmaceutical dispensing machine receives the information regarding each patient, what medicine he/she needs to receive, in which bed is located in which room on the floor mentioned in the scenario. The pharmaceutical dispensing machine automatically commences the preparation of a batch of pharmaceuticals for the morning medicines for the patients calculating the required time to prepare all the pharmaceuticals. The pharmaceutical dispensing machine can prepare the batched per room and/or per floor and/or in the order of beds in each room on the floor. The mechanical arm module begins to pick up pills from the drug elements and transport them to the designated envelope. Once all the required pills are in the envelope, the envelope is closed and the information of the patient is printed on the envelope. Then the envelope is transported into the tray in the pharmaceutical tote module. This process goes on until all the envelopes containing all the pills for all the patients of the floor are ready. Then the user and/or the nurse picks up the tray, ready to be delivered according to the predetermined order.

### Exemplary Interactions Between Pharmaceutical Dispensing Machines Located in the Same Location

In some embodiments, in one location there can be more than one pharmaceutical dispensing machine. In some embodiments, the pharmaceutical dispensing system optimizes the pharmaceutical dispensing activities according to the number and/or types of pharmaceutical dispensing devices in view of one or more pharmaceutical dispensing parameters (as will be further explained below). In some embodiments, pharmaceutical dispensing parameters are one or more of: users, patients, dedicated personnel, pharmaceuticals, dispensing schedules, pharmaceutical dispensing machines, location of one or more of users, patients, dedicated personnel, pharmaceuticals, pharmaceutical dispensing machines. In some embodiments, pharmaceutical dispensing parameters are correlated with one another to create correlation parameters. For example, using the exemplary pharmaceutical dispensing parameters “location of a patient” and “location of a specific pharmaceutical dispensing machine”, the correlation parameter could be: “the distance between a location of a patient and a location of a specific pharmaceutical dispensing machine” - which will provide the actual distance between the exemplary pharmaceutical dispensing parameters.

### Exemplary Real-Time Updating Between Pharmaceutical Dispensing Machines Regarding A Dispensing Action

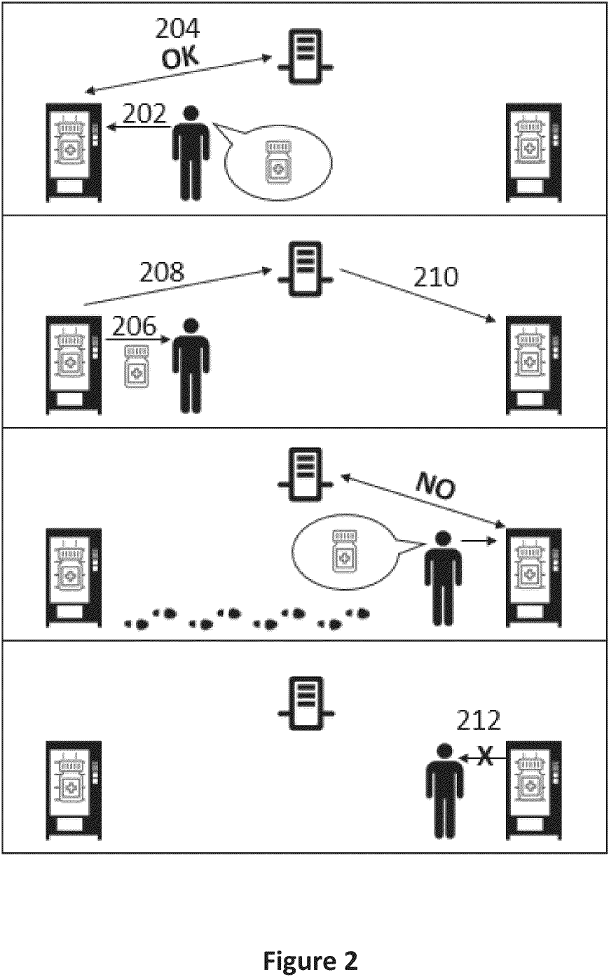

Referring now to FIG. 2, showing a schematic representation of an exemplary interaction between pharmaceutical dispensing machines and the pharmaceutical dispensing system, according to some embodiments of the invention. In some embodiments, a user/patient approaches one of the pharmaceutical dispensing machines located at the location to receive a pharmaceutical that was prescribed 202. The pharmaceutical dispensing machine contacts the server to check if it is allowed to dispense the pharmaceutical 204. For this example, the server confirms the dispensing of the pharmaceutical. In some embodiments, when the pharmaceutical dispensing machine dispenses a the pharmaceutical to the patient 206, the pharmaceutical dispensing machine sends a notification to the server 208, which then updates all pharmaceutical dispensing machines in the location 210. In some embodiments, updating all pharmaceutical dispensing machines regarding the dispensing action causes a change in behavior in all pharmaceutical dispensing machines. For example, if one pharmaceutical dispensing machine dispensed a certain pharmaceutical 206 to a specific patient, this will cause all other pharmaceutical dispensing machines to block any further dispensing of the same pharmaceutical to the same patient 212. In some embodiments, the pharmaceutical dispensing machines will block the dispensing of pharmaceuticals that can counteract the previous dispensed pharmaceutical. In some embodiments, the actions that a pharmaceutical dispensing machine can perform due to a dispensing event are one or more of:
Blocking further dispensing of the same pharmaceutical
Amending subsequent time of dispensing of pharmaceuticals
Amending type of subsequent pharmaceutical to be dispensed

In some embodiments, a potential advantage of real-time update of the pharmaceutical dispensing machines is that it potentially safeguard the well-being of the patients by actively monitoring and policing the pharmaceutical dispensing activities in the facility.

### Exemplary Pharmaceutical Dispensing Requirements

In some embodiments, pharmaceutical dispensing requirements are one or more of: optimal allocation of the workload between pharmaceutical dispensing machines, optimal division of pharmaceuticals between pharmaceutical dispensing machines according to pharmaceutical dispensing sub-requirements (see below).

### Exemplary Correlation Parameters Used for Allocating the Load of Work Between Pharmaceutical Dispensing Machines

In some embodiments, when two or more pharmaceutical dispensing devices are located at the same facility, the system divides the load of work between the devices according to one or more correlation parameters. In some embodiments, the parameters are:
Distance of the pharmaceutical dispensing machine from the patients.
Inventory of each pharmaceutical dispensing machine.
Pharmaceutical dispensing schedules.
Combination of distance of the pharmaceutical dispensing machine from a subgroup of patients and the inventory of pharmaceuticals in the same pharmaceutical dispensing machine.
Combination of pharmaceutical dispensing schedules and the inventory of pharmaceuticals in the pharmaceutical dispensing machines.

### Exemplary Division of Pharmaceuticals Between Pharmaceutical Dispensing Machines

In some embodiments, pharmaceuticals are divided between pharmaceutical dispensing machines located in the same location. In some embodiments, the pharmaceutical dispensing requirements that dictate the allocation of certain pharmaceuticals in a specific machine use one or more of the following correlation parameters:
Allocating pharmaceuticals in proximity to patients
Allocating specific pharmaceuticals in proximity to a relevant subset of patients that require said specific pharmaceuticals
Allocating pharmaceuticals in anticipation of needs
Allocating pharmaceuticals in anticipation of problems
Allocating pharmaceuticals according to frequency of requests
Allocating pharmaceuticals according to type of pharmaceutical
Allocating pharmaceuticals in view of known users
Allocating pharmaceutical according to real-time and/or historical prescription information
Allocating ready-to-be-dispensed envelopes between pharmaceutical dispensing machines
Allocating pharmaceuticals according to the expected location of the user
Allocating pharmaceuticals according to pharmaceutical dispensing schedules.
Allocating pharmaceuticals according to a level of disturbance that will cause dispensing from a certain pharmaceutical dispensing machine, where the disturbance is to one or more of a user and a pharmaceutical dispensing schedule.

### EXEMPLARY EMBODIMENTS OF ALLOCATION

### Allocating Pharmaceuticals in Proximity to Patients

In some embodiments, the large pharmaceutical dispensing machine 102 is located in a location, possibly not in proximity to any specific group of patients, this is to allow the major pharmaceutical packaging and dispensing process to be performed for all patients in the location. In some embodiments, other machines, for example small pharmaceutical dispensing machines 102b, are positioned in key locations according to the proximity to the patients. For example, one in each floor of patients that will spend a long period of time in the facility. In some embodiments, the content of type of pharmaceuticals in each pharmaceutical dispensing machine may be generic, for example, over the counter pharmaceuticals, pain management pharmaceuticals, generic IV’s, etc.

### Allocating Specific Pharmaceuticals in Proximity to a Relevant Subset of Patients That Require Said Specific Pharmaceuticals

In some embodiments, pharmaceutical dispensing machines are located in proximity to specific locations, which a relevant subset of patient frequent. For example, in proximity to an orthopedic department, where patients come to visit specialized orthopedic physicians, there will be a pharmaceutical dispensing machine containing mostly pharmaceuticals related to orthopedics, for example pain management creams and/or pills.

### Allocating Pharmaceuticals in Anticipation of Needs

In some embodiments, in anticipation of needs, for example, flu season, pharmaceuticals that are usually prescribed during this period will be allocated in the pharmaceutical dispensing machines that are located in the different locations of the facility. Furthermore, in some embodiments, in anticipation of overload requirement of a specific pharmaceutical, the pharmaceutical dispensing machines are instructed to limit the dispensing of the specific pharmaceutical and optionally provide alternatives.

### Allocating Pharmaceuticals in Anticipation of Problems

In some embodiments, if for example, the large pharmaceutical dispensing machine 102 is scheduled for maintenance, the pharmaceutical dispensing system divides the pharmaceutical dispensing labor between all the pharmaceutical dispensing machines in the location.

### Allocating Pharmaceuticals According to Frequency of Requests

In some embodiments, the pharmaceutical dispensing system monitors the frequency of requests for the pharmaceuticals. In some embodiments, when one or more pharmaceuticals are frequently requested from one location, the system increases the delivery of those pharmaceuticals to the relevant pharmaceutical dispensing machine.

### Allocating Pharmaceuticals According to Type of Pharmaceutical

In some embodiments, the pharmaceutical dispensing system monitors the type of pharmaceuticals provided to a specific location. In some embodiments, when one or more types of pharmaceuticals are frequently requested from one location, the system increases the delivery of those types of pharmaceuticals to the relevant pharmaceutical dispensing machine.

### Allocating Pharmaceuticals in View of Known Users

In some embodiments, pharmaceuticals dispensing machines located near a defined known subset of patients are filled with the specific pharmaceuticals needed for those specific defined subset of patients, in addition to other pharmaceuticals that are stored in the pharmaceutical dispensing machine. For example, patients optionally staying in a hospital for a known period of time, where their location optionally stays the same for that period of time, specific pharmaceuticals are stored in pharmaceutical dispensing machines near them, according to their needs and/or their schedule. In some embodiments, the choosing of which pharmaceutical dispensing machine will be used for the defined known subset of patients is the distance between the pharmaceutical dispensing machine and the defined known subset of patients.

### Allocating Pharmaceutical According to Real-time And/or Historical Prescription Information

In some embodiments, every single pharmaceutical dispensing action is recorded in the server. In some embodiments, analysis of the pharmaceutical dispensing actions (prescriptions and/or actual dispensing) is performed on the raw data in order to extrapolate relevant information that will be used for the allocation and/or reallocation of pharmaceuticals between the pharmaceutical dispensing machines in the location. For example, when a specific pharmaceutical is requested near a specific location over a period of time, the pharmaceutical dispensing system updates the contents of the nearest pharmaceutical dispensing machine to include that specific pharmaceutical. In some embodiments, a period of time may be a week, a month, a year, different periods over the year, and any combination thereof.

### Allocating Ready-to-be-Dispensed Envelopes Between Pharmaceutical Dispensing Machines

In some embodiments, the pharmaceutical dispensing machines final product is an envelope (or other transport container) comprising the necessary pharmaceuticals for a specific user. In some embodiments, ready-to-be-dispensed envelopes are moved from one pharmaceutical dispensing machine to other, before being dispensed to the final patient. For example, the big pharmaceutical dispensing machine in the basement prepares all the envelopes for the patients in the 3^{rd} floor of the building. In some embodiments, the envelopes are moved to a small pharmaceutical dispensing machine located in the 3^{rd} floor before they are finally dispensed to the patients. In some embodiments, the movement of ready-to-be-dispensed envelopes is performed by someone from the personnel of the location. In some embodiments, the movement of ready-to-be-dispensed envelopes is performed by a specialized machine that carries the envelopes from one machine to another, for example, a robot or specialized transportation conveyors between machines.

### Allocating Pharmaceuticals According to the Expected Location of the User

In some embodiments, in view of an expected known location of the user, the pharmaceutical dispensing system allocates the necessary pharmaceuticals to be dispensed to the closest pharmaceutical dispensing machine. In some embodiments, the location of the pharmaceutical dispensing machine that is chosen to perform the dispensing is sent to the dedicated personnel and/or to the user. In some embodiments, if the expected location of the user changes and the user and/or the dedicated personnel cannot access the location of the chosen pharmaceutical dispensing machine, the pharmaceutical dispensing system will indicate the closest pharmaceutical dispensing machine to the actual location of the user and/or personnel, which comprises the pharmaceuticals needed to be dispensed. In some embodiments, once the user and/or the dedicated personnel have received the pharmaceuticals, the system updates the database on the dispensing action for future reference.

In some embodiments, in either of the abovementioned correlation parameters, the pharmaceuticals under consideration can be one or more of scheduled pharmaceuticals to be dispensed, unscheduled pharmaceuticals potentially to be dispensed, a combination thereof, pharmaceuticals that are needed to be dispensed as part of a multi-daily routine (for example, morning, noon and evening).

### Exemplary Computational Logistics System Comprising Modules and Simulations

In some embodiments, the system utilizes a computational logistics system comprising one or more computational logistics modules to perform one or more of: assessing, correlating, evaluating, activating - one or more interactions between pharmaceutical dispensing machines located in the same location. In some embodiments, the one or more computational logistics modules utilize one or more of neural networks, machine learning and simulations.

In some embodiments, the computational logistics module receives information from the pharmaceutical dispensing system and/or real-time information from external sources (i.e. manual inputs, the internet, the news, the weather, etc.) and create specific models of the next cycle of pharmaceutical dispensing processes, including but not exclusively, the distribution of pharmaceuticals between the one or more pharmaceutical dispensing machines located in the same location. In some embodiments, the computational logistics module utilizes one or more of neural networks, Machine Learning (ML), simulations, etc. In some embodiments, the models use best case scenarios to run, for example, simulations, which mean there are no technical problems, no logistics problems, no inventory problems, etc., and a model of the pharmaceutical dispensing process in the one or more pharmaceutical dispensing machines located in the same location is performed from the beginning to the end of the pharmaceutical dispensing process. In some embodiments, the models use worst case scenarios, to run, for example, simulations, which mean there are technical problems, there are logistics problems, and/or there are inventory problems, etc., and a model of the pharmaceutical dispensing process in the one or more pharmaceutical dispensing machines located in the same location, together with the response to those problems, is performed from the beginning to the end of the pharmaceutical dispensing process. In some embodiments, the models use the actual status of the one or more pharmaceutical dispensing machines located in the location and the information/correlation parameters as disclosed herein to simulate the pharmaceutical dispensing process from the beginning to end. In some embodiments, at the end of each simulation, each simulation is provided with a score, which is then used for the evaluation of the pharmaceutical dispensing process. In some embodiments, the time needed to finish a best case scenario pharmaceutical dispensing process is compared with the time needed to finish a worst case scenario pharmaceutical dispensing process to evaluate, by the pharmaceutical dispensing action module, if steps are to be taken in order to ensure the dispensing, for example, anticipating the beginning of the pharmaceutical dispensing process by the pharmaceutical dispensing machine, dispatching a technician, preparing a warehouse to dispatch pharmaceuticals, requesting a self-check analysis of a specific pharmaceutical dispensing machine, redistributing pharmaceuticals between pharmaceutical dispensing machines located in the same location, etc.

In some embodiments, the results provided by the models are responses to direct inquiries, for example, “can the pharmaceutical dispensing process be finished in time for delivery according to the pharmaceutical dispensing schedule?”, or “which pharmaceutical dispensing service ensures finishing the pharmaceutical dispensing process on time?”. In some embodiments, the computational logistics model module is configured to change the parameters in order to provide at least one possible solution so the pharmaceutical dispensing process can be achieved in the pharmaceutical dispensing schedule.

In some embodiments, models can be performed on any single part of the pharmaceutical logistical dispensing process, for example, the arrival of pharmaceuticals from a supplier, transporting pharmaceuticals from a warehouse to a long term care facility, a specific pharmaceutical dispensing machine, etc.

In some embodiments, the models are performed automatically in a periodical manner. In some embodiments, the models are performed manually by an administrator of the pharmaceutical dispensing system. In some embodiments, models are performed in relation to a specific single machine in a location. In some embodiments, models are performed for entire pharmaceutical ecosystems, and anything in between an ecosystem to a specific single pharmaceutical dispensing machine.

In some embodiments, the information used to run the simulations on the models is updated after every cycle performed by the pharmaceutical logistic tracking module, after every pharmaceutical dispensing cycle performed by the logistic operational system, and after manual insertion of information from an authorized personnel from the pharmaceutical dispensing system.

In some embodiments, models are run in the time between dispensing cycles.

### Exemplary Actions Performed in View of Run Models

In some embodiments, after simulations have been run, the system is configured to analyze the information and provide actionable instructions over the pharmaceutical dispensing system in order to guarantee the dispensing of pharmaceuticals according to the main directive of the pharmaceutical dispensing system - ensuring the provision of medical care to the patients -optionally also in view of a potential optimal distribution of the work load and the pharmaceuticals according to the parameters as disclosed herein. In some embodiments, the system is equipped with deep learning algorithms, which enable the system to learn from each situation/simulation, actionable instruction and final outcome of the dispensing due to the actionable instruction.

In some embodiments, actionable instructions are provided in order to schedule the dispensing according to the window of foreseeable failures, the possible responses to said failures and the positive outcome of the pharmaceutical dispensing process. In some embodiments, the pharmaceutical dispensing process is scheduled according to the Mean time between failures (MTBF). In some embodiments, the MTBF is calculated by the information provided by the pharmaceutical logistic tracking module and the pharmaceutical logistic model module.

### Exemplary Main Directive of the Pharmaceutical Dispensing System

In prior art pharmaceutical dispensing systems, the main directive is the arrival of pharmaceuticals to the patients. While this directive is also part of the directives governing the actions of the pharmaceutical dispensing system of the present invention, a higher main directive governs above all directives and instructions: guaranteeing the reliable provision of medical care to the patients - optionally also in view of a potential optimal distribution of the work load and/or the actual location of the pharmaceuticals according to the parameters as disclosed herein.

In some embodiments, the system provides instructions not based on the directive that pharmaceuticals must arrive at their destination, but rather what is the medical care needed by the patient. In some embodiments, to this scope, the system is in continuous communication with two or more medical sources: a medical pharmaceutical database and medical professional staff (i.e. physicians, nurses, pharmacists), which provide medical input in real-time. In some embodiments, the system is also in communication with the patient itself to monitor and receive real-time feedback from the patient.

### Allocating Pharmaceuticals According to Pharmaceutical Dispensing Schedules

In some embodiments, pharmaceutical dispensing schedules influence the distribution of pharmaceuticals between pharmaceutical dispensing machines in the same location. For example, when one machine is scheduled to prepare a plurality of pharmaceuticals to be dispensed, thereby being occupied doing that and cannot and/or should not be disturbed so as to meet the dispensing schedule, pharmaceuticals that are expected to be requested and/or are in high request and/or are essential for urgent matters will be distributed to a second pharmaceutical dispensing machine in order to avoid possible disturbances in the dispensing process occurring in the first pharmaceutical dispensing machine.

### Allocating Pharmaceuticals According to a Level of Disturbance/Disruption That Will Cause Dispensing from a Certain Pharmaceutical Dispensing Machine, Where the Disturbance is to a User and/or to a Pharmaceutical Dispensing Schedule

In some embodiments, pharmaceuticals that are expected to be dispensed to a specific patient population and/or to a specific patient will be distributed to pharmaceutical dispensing machines that are located the closest possible to the expected location of the patients, thereby potentially reducing the disturbance/disruption of the patients to a minimum. In some embodiments, levels of disturbance/disruption are measured according to distance, for example the distance that the user/patient will need to do from a location (i.e. physician’s office or patient’s room) to the location of the pharmaceutical dispensing machine. In some embodiments, the lowest distance is from about 0 meters to about 100 meters. Optionally from about 0 meters to about 75 meters. Optionally from about 0 meters to about 50 meters. In some embodiments, levels of disturbance/disruption are measured according to time, for example the time that will take the user/patient to arrive from a location (i.e. physician’s office or patient’s room) to the location of the pharmaceutical dispensing machine. In some embodiments, the lowest time is from about 1 minute to about 60 minutes. Optionally from about 30 seconds to about 30 minutes. Optionally from about 10 seconds to about 10 minutes.

In some embodiments, when a pharmaceutical dispensing machine must prepare one or more batches of pharmaceuticals to one or more users/patients in order to meet a specific pharmaceutical dispensing schedule, the preparation of the batches of pharmaceuticals requires a certain period of time. In some embodiments, the pharmaceutical dispensing machine is configured to begin the pharmaceutical dispensing process in time to meet the pharmaceutical dispensing schedule. In some embodiments, if the pharmaceutical dispensing machine is interrupted to dispense a non-scheduled pharmaceutical, the pharmaceutical dispensing schedule would be potentially not met due to the disruption of the pharmaceutical dispensing process. In some embodiments, the systems allocates the pharmaceuticals according to an expected potential level of disruption/disturbance of the dispensing of a non-scheduled pharmaceutical by the pharmaceutical dispensing machine if and when would be interrupted to dispense the non-scheduled pharmaceutical. For example, non-scheduled pharmaceuticals having a higher possibility to be requested during the day comprise a higher level of disruption compared to a non-scheduled pharmaceutical that is rarely dispensed. In some embodiments, each non-scheduled pharmaceutical is scored according a level of disruption/disturbance to the pharmaceutical dispensing schedule and those comprising a higher level of potential disruption/disturbance will be allocated in a second pharmaceutical dispensing machine. In some embodiments, the level of disruption/disturbance is calculated in percentage, where from about 0% to about 25% is considered low level of disruption/disturbance, from about 25% to about 40% is considered medium level of disruption/disturbance, from about 40% to about 60% is considered high level of disturbance, from about 60% to about 80% is considered very high level of disruption/disturbance, from about 80% to about 100% is considered extremely high disruption/disturbance.

### Exemplary Scoring of Pharmaceuticals to Be Used in Optimization of Distribution Of Pharmaceuticals in Two or More Pharmaceutical Dispensing Machines Located in a Same Location

In some embodiments, a first exemplary configuration of pharmaceutical dispensing machines in the same location comprises at least one first pharmaceutical dispensing machine, configured to prepare a plurality of batches of pharmaceuticals to a plurality of patients/users according to a determined pharmaceutical dispensing schedule; and at least one second pharmaceutical dispensing machine located in proximity to the personnel and/or the patients/users.

As mentioned above, in some embodiments, each pharmaceutical dispensing device comprises a certain storage capacity. In some embodiments, the storage capacity depends on one or more of the number of pharmaceutical containers in the device, the size of the pharmaceutical containers, the size of the pharmaceuticals that will dictate the number of pharmaceuticals that can be storage in a pharmaceutical container and the type of pharmaceutical (pills, liquid, and powder).

In some embodiments, the at least one first pharmaceutical dispensing machine comprises a big capacity for storing pharmaceuticals, for example from about 100 to about 200 different types of pharmaceuticals. Optionally form about 100 to about 500 different types of pharmaceuticals. In some embodiments, the at least one first machine is accessible only to the personnel of the facility and, optionally, is located in a secured room. In some embodiments, the at least one second pharmaceutical dispensing machine comprises a smaller capacity for storing pharmaceuticals in comparison to the at least one first pharmaceutical dispensing machine, for example from about 50 to about 100 different types of pharmaceuticals. In some embodiments, the at least one second pharmaceutical dispensing machine is accessible to anyone (personnel, patients, users), which can access the machine at any time. In some embodiments, it is required to allocate all the necessary pharmaceuticals between the at least one first pharmaceutical dispensing machine and the at least one second pharmaceutical dispensing machine. In order to facilitate the explanations, the at least one first pharmaceutical dispensing machine will be referred as “big pharmaceutical dispensing machine”, while the at least one second pharmaceutical dispensing machine will be referred as “small pharmaceutical dispensing machine”.

Potential considerations in the decision of the allocation of pharmaceuticals include for example the following:

The at least one big pharmaceutical dispensing machine is constricted to the pharmaceutical dispensing schedule, which must be met.

The limitation in storage of the at least one small pharmaceutical dispensing machine.

The location of the at least one small pharmaceutical dispensing machine relative to patients/users/personnel.

Any and/or all of the aforementioned parameters disclosed in “Exemplary division of pharmaceuticals between pharmaceutical dispensing machines”.

In some embodiments, pharmaceuticals needed to be stored in two or more of the aforementioned pharmaceutical dispensing machines are divided in at least two groups. In some embodiments, one group are pharmaceuticals that are scheduled to be dispensed in batches according to a pharmaceutical dispensing schedule. In some embodiments, the second group are pharmaceuticals that are not scheduled to be dispensed in batches according to a pharmaceutical dispensing schedule.

In some embodiments, pharmaceuticals of the second group are subjected to a scoring process in order to decide where they should be stored. In some embodiments, the scoring relates to a degree of potential disruption caused to the pharmaceutical dispensing schedule if the dispensing of pharmaceuticals from the second group would be dispensed by the at least one big pharmaceutical dispensing machine. In some embodiments, the scoring utilizes data from the abovementioned run models. In some embodiments, the scoring is performed utilizing one or more of real-time data, historical data, any or all of the abovementioned exemplary correlation parameters, any or all of the abovementioned exemplary pharmaceutical dispensing requirements. In some embodiments, the physical storage limitations of the different machines is a parameter included in the scoring. In some embodiments, after the pharmaceuticals of the second group have been scored, those that scored with the least degree of potential disruption to the pharmaceutical dispensing schedule will be placed in the at least one big pharmaceutical dispensing machine, while those pharmaceuticals that scored with the highest degree of potential disruption will be placed in the at least one small pharmaceutical dispensing machine. In some embodiments, the at least one small pharmaceutical dispensing machine will receive scored pharmaceuticals until said at least one small pharmaceutical dispensing machine is full.

In some embodiments, factors that contribute to the allocation of pharmaceuticals, for example, pharmaceuticals that need to be dispensed together with other pharmaceuticals will be allocated together albeit of their score.

In some embodiments, alternatively or additionally, factors that contribute to the scoring relate to a degree of potential disruption caused to the user that needs to pick up the pharmaceuticals if the dispensing of pharmaceuticals from the second group would be dispensed by the at least one big pharmaceutical dispensing machine. In some embodiments, the scoring utilizes data from the abovementioned run models. In some embodiments, the scoring is performed utilizing one or more of real-time data, historical data, physical location of the user, expected location of the user at an expected time of dispensing, any or all of the abovementioned exemplary correlation parameters, any or all of the abovementioned exemplary pharmaceutical dispensing requirements. In some embodiments, the physical storage limitations of the different machines is a parameter included in the scoring. In some embodiments, after the pharmaceuticals of the second group have been scored, those that scored with the least degree of potential disruption to the user will be placed in the at least one big pharmaceutical dispensing machine, while those pharmaceuticals that scored with the highest degree of potential disruption to the user will be placed in the at least one small pharmaceutical dispensing machine (because is closer to the user). In some embodiments, the at least one small pharmaceutical dispensing machine will receive scored pharmaceuticals until said at least one small pharmaceutical dispensing machine is full. In some embodiments, the at least one small pharmaceutical dispensing machine is relatively closer to the user than the at least one big pharmaceutical dispensing machine.

In some embodiments, alternatively or additionally, factors that contribute to the scoring relate to a degree of potential urgency of dispensing of specific pharmaceuticals. In some embodiments, urgency is measured by the time required to receive the pharmaceutical so a patient/user does not die and/or is not further injured. In some embodiments, urgency is measured in minutes, optionally in hours. In some embodiments, potential urgent pharmaceuticals will be allocated in the at least one small pharmaceutical dispensing machine. In some embodiments, the at least one small pharmaceutical dispensing machine is relatively closer to the user that the at least one big pharmaceutical dispensing machine. In some embodiments, the urgency scoring utilizes data from the abovementioned run models. In some embodiments, the urgency scoring is performed utilizing one or more of real-time data, historical data, physical location of the user, expected location of the user at an expected time of dispensing, any or all of the abovementioned exemplary correlation parameters, any or all of the abovementioned exemplary pharmaceutical dispensing requirements.

In some embodiments, alternatively or additionally, factors that contribute to the scoring relate to frequency of dispensing of specific pharmaceuticals. In some embodiments, pharmaceuticals having high frequency of dispensing will be allocated in the at least one small pharmaceutical dispensing machine. In some embodiments, the at least one small pharmaceutical dispensing machine is relatively closer to the user that the at least one big pharmaceutical dispensing machine. In some embodiments, the frequency scoring utilizes data from the abovementioned run models. In some embodiments, the frequency scoring is performed utilizing one or more of real-time data, historical data, physical location of the user, expected location of the user at an expected time of dispensing, any or all of the abovementioned exemplary correlation parameters, any or all of the abovementioned exemplary pharmaceutical dispensing requirements.

In some embodiments, alternatively or additionally, factors that contribute to the scoring relate to specific subpopulations of users, their proximity to the pharmaceutical dispensing machine and the dispensing of specific pharmaceuticals to the specific subpopulation. In some embodiments, relevant pharmaceuticals to a specific subpopulation of users will be allocated in the at least one small pharmaceutical dispensing machine. In some embodiments, the at least one small pharmaceutical dispensing machine is relatively closer to the user that the at least one big pharmaceutical dispensing machine. In some embodiments, the scoring utilizes data from the abovementioned run models. In some embodiments, the scoring is performed utilizing one or more of real-time data, historical data, physical location of the subpopulation of users, expected location of the subpopulation of users at an expected time of dispensing, any or all of the abovementioned exemplary correlation parameters, any or all of the abovementioned exemplary pharmaceutical dispensing requirements.

In some embodiments, alternatively or additionally, factors that contribute to the scoring relate to the anticipation of needs of specific pharmaceuticals. In some embodiments, relevant pharmaceuticals to a specific need (for example flu medicine) will be allocated in the at least one small pharmaceutical dispensing machine. In some embodiments, the at least one small pharmaceutical dispensing machine is relatively closer to the users that the at least one big pharmaceutical dispensing machine. In some embodiments, the scoring utilizes data from the abovementioned run models. In some embodiments, the scoring is performed utilizing one or more of real-time data, historical data, physical location of the users expected to need the pharmaceuticals, expected location of the users at an expected time of dispensing, any or all of the abovementioned exemplary correlation parameters, any or all of the abovementioned exemplary pharmaceutical dispensing requirements.

In some embodiments, alternatively or additionally, factors that contribute to the scoring relate to the anticipation of problems in the pharmaceutical dispensing system, for example, a scheduled powering down of the at least one big pharmaceutical dispensing machine during a defined period of time. In some embodiments, relevant pharmaceuticals will be allocated in the at least one small pharmaceutical dispensing machine to provide backup to while the other machine is off-line. In some embodiments, the scoring utilizes data from scheduled maintenance of the pharmaceutical dispensing machines.

### Exemplary System With Two or More Small Pharmaceutical Dispensing Machines Only

In some embodiments, a second exemplary configuration of pharmaceutical dispensing machines in the same location comprises two or more small pharmaceutical dispensing machines, having less storage capacity, configured to store and prepare a plurality of batches of pharmaceuticals to a plurality of patients/users according to a determined pharmaceutical dispensing schedule and configured to store and dispense non-scheduled pharmaceuticals. In some embodiments, the small machines are located in proximity to the personnel and/or the patients/users. In some embodiments, when compared to the first exemplary configuration, the difference is that there are only small pharmaceutical dispensing machines located in proximity to the personnel and/or the patients/users, and there is no big pharmaceutical dispensing machine. In some embodiments, each small pharmaceutical dispensing machine is responsible to store, prepare and dispense pharmaceuticals to be dispensed in batches. In some embodiments, each small pharmaceutical dispensing machine prepares batches for those users/patients located in close proximity to the machine. For example, a pharmaceutical dispensing machine is located in the third floor of a hospital. In some embodiments, that pharmaceutical dispensing machine will prepare the batches of pharmaceuticals to be dispensed for all the users/patients of that floor. In some embodiments, the two or more small pharmaceutical dispensing machines work together as a single pharmaceutical dispensing system where the workload of the entire facility is coordinated and divided between the machines.

In some embodiments, optionally, each small pharmaceutical dispensing machine is further stored with pharmaceuticals that are dispensed when necessary, meaning not related to those pharmaceuticals that are dispensed according to a determined pharmaceutical dispensing schedule. In some embodiments, optionally, the non-scheduled pharmaceuticals located in each machine are selected according to type of population located near each small pharmaceutical dispensing machine. For example, a small pharmaceutical dispensing machine located in an orthopedic department of a hospital will be provided with non-scheduled pharmaceuticals related to the orthopedic field, while a small pharmaceutical dispensing machine located in a cardiologic department of the hospital will be provided with non-scheduled pharmaceuticals related to the cardiologic field.

### Exemplary Allocation of Pharmaceuticals According to the Number of Pharmaceutical Containers in the Device, the Size of the Pharmaceutical Containers, the Size of the Pharmaceuticals That Will Dictate the Number of Pharmaceuticals That Can be Storage in a Pharmaceutical Container and the Type of Pharmaceutical (Pills, Liquid, and Powder)

In some embodiments, the considerations of the distribution of pharmaceuticals include also the configuration of the pharmaceutical containers in the pharmaceutical dispensing devices. For example, the number of pharmaceutical containers in the device, the size of the pharmaceutical containers, the size of the pharmaceuticals that will dictate the number of pharmaceuticals that can be storage in a pharmaceutical container and the type of pharmaceutical (pills, liquid, and powder). In some embodiments, optionally or additionally, the frequency of dispensing is also taken under consideration with the configuration of the pharmaceutical containers in the pharmaceutical dispensing devices. For example, if a certain pharmaceutical is regularly dispensed but it takes a lot of space in the pharmaceutical dispensing machine because it requires a big pharmaceutical container, even though it could be allocated in a small pharmaceutical dispensing machine because of its dispensing frequency, that certain pharmaceutical will be allocated in the big pharmaceutical dispensing machine in order to save space for other pharmaceuticals in the small pharmaceutical dispensing machine.

In some embodiments, the storage capacity of a pharmaceutical dispensing device is measured according to potential period of the total time that a pharmaceutical dispensing schedule can be fulfilled by the device. For example, a pharmaceutical dispensing machine comprises enough space to fulfil three consecutive days of dispensing according to a determined schedule the storage capacity is measured in days of dispensing. In some embodiments, the storage capacity is measured in minutes, optionally in hours, optionally in days, optionally in weeks.

### Exemplary Use of Special Pharmaceutical Containers

In some embodiments, the pharmaceutical dispensing devices are required to store pharmaceuticals in special conditions (temperature, humidity) and/or in special forms of delivery (powder, liquid). In some embodiments, dedicated pharmaceutical units can be added a priori to the pharmaceutical dispensing devices in order to allow the storage and dispensing of those pharmaceuticals that required special conditions and/or special forms of delivery. In some embodiments, the use of such dedicated containers and the space required to use them in the pharmaceutical dispensing devices are taken under consideration when evaluating the optimal distribution of pharmaceuticals between pharmaceutical dispensing devices. For example, if a certain pharmaceutical requires cold storage conditions and the addition of a special unit to a small pharmaceutical dispensing machine will require the use of space that could be better used to store more pharmaceuticals, then the pharmaceuticals will be sent to the big pharmaceutical dispensing machine.

### Exemplary Transportation of Ready to Be Dispensed Pharmaceuticals From One Pharmaceutical Dispensing Machine to Another

In some embodiments, each pharmaceutical dispensing machine is configured to prepare individual packages (either alone or in batches) containing one or more different pharmaceuticals needed to be dispensed to one or more users/patients according to a determined pharmaceutical dispensing schedule. In some embodiments, each individual package is for a specific user/patient.

In some embodiments, the two or more pharmaceutical dispensing machines, whether they are big or small pharmaceutical dispensing machines, are configured to transfer ready-to-be-dispensed packages between each other. In some embodiments, the transfer of ready-to-be-dispensed packages is perform with a net of conveyors and/or pneumatic tubes and/or dedicated autonomous mobile robots connecting the pharmaceutical dispensing machines.

### Exemplary Method of Notification of Divided Pharmaceutical Containers

In some embodiments, as stated above, personalized pharmaceutical dispensing containers are prepared for each individual user/patient, each container comprising one or more of different pharmaceuticals. In some embodiments, when one pharmaceutical dispensing machine is lacking one or more pharmaceuticals needed to be dispensed to a single user/patient but another pharmaceutical dispensing machine has them, the system will generate two or more personalized pharmaceutical dispensing containers for the same user/patient. In some embodiments, on the label of the first personalized pharmaceutical dispensing container will be a dedicated message containing information regarding the location of the second personalized pharmaceutical dispensing container. In some embodiments, the first personalized pharmaceutical dispensing container is the first one that will be picked up from a pharmaceutical dispensing machine.

### Exemplary Division of Pharmaceuticals According to Availability of Dedicated Personnel

In some embodiments, the distribution of pharmaceuticals takes under consideration the availability of dedicated personnel for picking up pharmaceuticals for the users/patients. For example, if a certain department comprising a first pharmaceutical dispensing machine lacks personnel able to pick up the pharmaceuticals for the users/patients, the pharmaceuticals will be distributed to a different pharmaceutical dispensing machine where there is dedicated personnel instructed to pick up the pharmaceuticals and are able to deliver the pharmaceuticals to the users/patients.

### Exemplary Methods

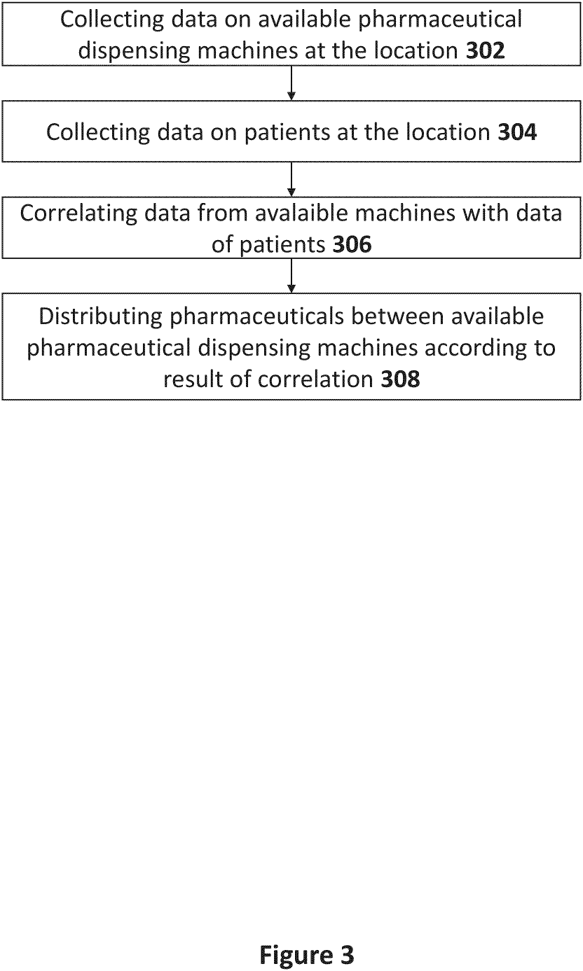

Referring now to FIG. 3, showing a flowchart of an exemplary method of organization of pharmaceuticals between one or more pharmaceutical dispensing devices, according to some embodiments of the invention. In some embodiments, the pharmaceutical dispensing system operates following the following actions.

In some embodiments, the system begins by collecting the relevant data regarding the availability of pharmaceutical dispensing devices at the location 302. In some embodiments, collection of data on pharmaceutical dispensing devices includes the type of pharmaceutical dispensing device, the number of pharmaceutical dispensing devices in the location, historical maintenance data related to each pharmaceutical dispensing device and future scheduled maintenance of the pharmaceutical dispensing devices. In some embodiments, the pharmaceutical dispensing devices can be anyone of the devices as mentioned above (102a-e).

In some embodiments, the system then collects the data related to the patients in the location 304. In some embodiments, assessment of the patient data includes one or more of: quantity of patients, type of pharmaceuticals taken by the patients, dispensing schedule of pharmaceuticals, relevant input provided by personnel and/or system, for example: influenza period, coronavirus isolation regime, etc.

In some embodiments, the system then correlates between the patient data and the pharmaceutical dispensing device data in the location 306. In some embodiments, correlation of data includes one or more of correlating between the location of patients and the location of pharmaceutical dispensing devices, correlating between the type of pharmaceuticals used by patients and their location.

In some embodiments, the system then distributes pharmaceuticals between the available pharmaceuticals dispensing machines according to the results of the correlation between the patient data and the pharmaceutical dispensing device data in the location 308.

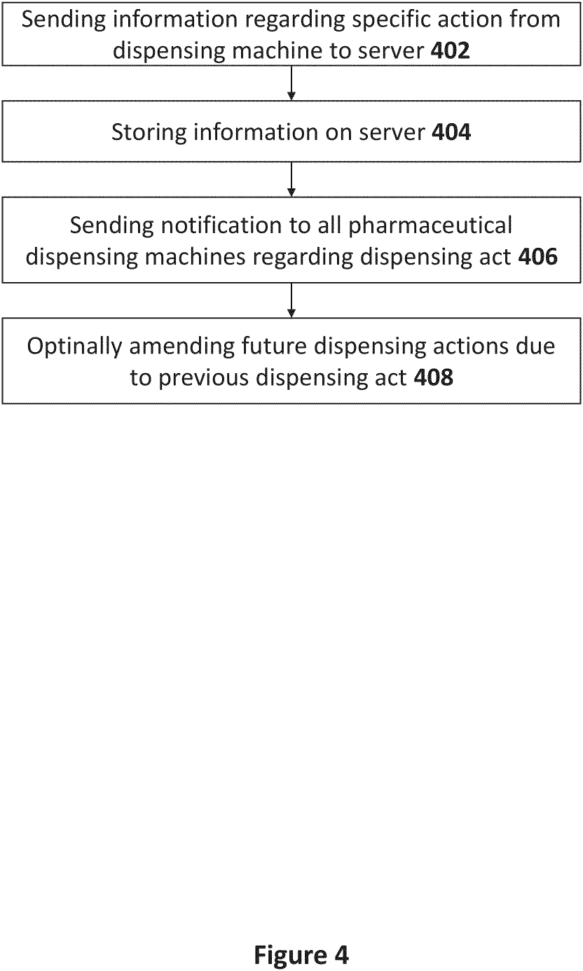

Referring now to FIG. 4, showing a flowchart of an exemplary method of interaction between pharmaceutical dispensing machines and a pharmaceutical dispensing system, according to some embodiments of the invention. In some embodiments, information from one or more pharmaceutical dispensing machine regarding one or more actions, for example dispensing of a specific pharmaceutical to a specific user, is sent to the server of the pharmaceutical dispensing system 402. In some embodiments, the information is stored in the server 404. In some embodiments, when the dispensing process is complete, the server updates all pharmaceutical dispensing machines regarding the dispensing act 406. In some embodiments, the information regarding the pharmaceutical dispensing act optionally causes a change in the next pharmaceutical dispensing action 408, for example blocking further dispensing of the same pharmaceutical, amending subsequent time of dispensing of pharmaceuticals, amending type of subsequent pharmaceutical to be dispensed.

As used herein with reference to quantity or value, the term “about” means “within ± 20 % of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of distributing pharmaceuticals in a pharmaceutical dispensing system, said pharmaceutical dispensing system comprising at least one first pharmaceutical dispensing machine configured to store, package and dispense pharmaceuticals in batches according to a pharmaceutical dispensing schedule, at least one second pharmaceutical dispensing machine configured to store, package and dispense non-scheduled pharmaceuticals in response to at least one non-scheduled demand, said at least one second pharmaceutical dispensing machine comprising a limited pharmaceutical storage capacity which is less than a pharmaceutical storage capacity of said at least one first pharmaceutical dispensing machine, the method comprising:
distributing pharmaceuticals to be dispensed according to said pharmaceutical dispensing schedule to said at least one first pharmaceutical dispensing machine and distributing only a subset of non-scheduled pharmaceuticals to said at least one second pharmaceutical dispensing machine;
said distributing only a subset of non-scheduled pharmaceuticals to said at least one second pharmaceutical dispensing machine comprising determining said subset by:
(a) scoring each non-scheduled pharmaceutical by a degree of expected disruption caused to said pharmaceutical dispensing schedule by dispensing thereof by said at least one first pharmaceutical dispensing machine;
(b) finding a subset of said non-scheduled pharmaceuticals which reduces said expected disruption based on said scoring and also fits in said limited pharmaceutical storage capacity of said at least one second pharmaceutical dispensing machine.

2. The method according to claim 1, wherein said determining said subset of non-scheduled pharmaceuticals further comprises scoring said each non-scheduled pharmaceutical by a degree of expected disturbance caused to at least one user.

3. The method according to claim 2, wherein said at least one second pharmaceutical dispensing machine being closer to said at least one user than said at least one first pharmaceutical dispensing machine.

4. The method according to claim 1, wherein pharmaceuticals that are expected to be dispensed together are distributed to the same pharmaceutical dispensing machine albeit of their scoring.

5. The method according to claim 1, wherein said determining said subset of non-scheduled pharmaceuticals further comprises one or more of:
a. scoring each non-scheduled pharmaceutical by a degree of potential urgency of dispensing of non-scheduled pharmaceuticals;
b. scoring each non-scheduled pharmaceutical by frequency of dispensing of non-scheduled pharmaceuticals;
c. scoring each non-scheduled pharmaceutical by specific subpopulations of users, their proximity to said at least one second pharmaceutical dispensing machine and the past history data of dispensing of non-scheduled pharmaceuticals to said specific subpopulation;
d. scoring each non-scheduled pharmaceutical by an anticipation of needs of non-scheduled pharmaceuticals; and
e. scoring each non-scheduled pharmaceutical by an anticipation of problems in the pharmaceutical dispensing system.

6-8. (canceled)

9. The method according to claim 5, wherein said anticipation of needs are based on data from one or more of run models, real-time data, historical data, physical location of users expected to need the pharmaceuticals, expected location of users at an expected time of dispensing.

10. (canceled)

11. The method according to claim 5, wherein said anticipation of problems are based on data from one or more of scheduled maintenance, run models and historical data.

12. The method according to claims 1, wherein said at least one first pharmaceutical dispensing machine is further configured to store, package and dispense non-scheduled pharmaceuticals in response to at least one non-scheduled demand.

13. The method according to claim 1, wherein said at least one second pharmaceutical dispensing machine is further configured to store, package and dispense pharmaceuticals in batches according to a pharmaceutical dispensing schedule.

14. The method according to claim 1, wherein said dispense non-scheduled pharmaceuticals in response to at least one non-scheduled demand is done immediately after the at least one non-scheduled demand is received.

15. The method according to claim 1, wherein said expected disruption caused to said schedule is caused by disruption of a pharmaceutical dispensing process of said pharmaceuticals in batches; and
wherein said expected disruption of said pharmaceutical dispensing process of said pharmaceuticals in batches is caused by said dispensing of said non-scheduled pharmaceuticals in response to said at least one non-scheduled demand.

16. (canceled)

17. The method according to claim 1, further comprising dispensing said pharmaceuticals according to said schedule.

18. A pharmaceutical dispensing system, comprising:
a. at least one first pharmaceutical dispensing machine for storing, packing and dispensing one or more pharmaceuticals in batches according to a pharmaceutical dispensing schedule;
b. at least one second pharmaceutical dispensing machine for storing, packing and dispensing one or more non-scheduled pharmaceuticals in response to at least one non-scheduled demand;
wherein said one or more non-scheduled pharmaceuticals included in said at least one second pharmaceutical dispensing machine reduce expected disruption in said pharmaceutical dispensing schedule in comparison to including said one or more non-scheduled pharmaceuticals in said at least one first pharmaceutical dispensing machine.

19. The pharmaceutical dispensing system according to claim 18, wherein said one or more non-scheduled pharmaceuticals are chosen according to a scoring of each non-scheduled pharmaceutical by a degree of expected disturbance to at least one user, said chosen one or more non-scheduled pharmaceuticals being those that reduces said expected disturbance.

20. The pharmaceutical dispensing system according to claim 19, wherein said at least one second pharmaceutical dispensing machine being closer to said at least one user than said at least one first pharmaceutical dispensing machine.

21. The pharmaceutical dispensing system according to claim 18, wherein pharmaceuticals that are expected to be dispensed together are distributed to the same pharmaceutical dispensing machine albeit of their scoring.

22. The pharmaceutical dispensing system according to claim 18, wherein said one or more non-scheduled pharmaceuticals are chosen according to one or more of:
a. a scoring of each non-scheduled pharmaceutical by a degree of potential urgency of dispensing of non-scheduled pharmaceuticals, said chosen one or more non-scheduled pharmaceuticals being those higher degree of potential urgency;
b. a scoring of each non-scheduled pharmaceutical by a degree of frequency of dispensing of non-scheduled pharmaceuticals, said chosen one or more non-scheduled pharmaceuticals being those higher degree of frequency;
c. a scoring of each non-scheduled pharmaceutical by specific subpopulations of users, their proximity to said second pharmaceutical dispensing machine and the frequency of dispensing of said non-scheduled pharmaceuticals to said specific subpopulation, said chosen one or more non-scheduled pharmaceuticals being those dispensed by said specific subpopulations of users, which are the closest to said second pharmaceutical dispensing machine and having a higher degree of frequency;
d. a scoring of each non-scheduled pharmaceutical by a degree of anticipation of needs of non-scheduled pharmaceuticals, said chosen one or more non-scheduled pharmaceuticals being those with higher degree of anticipation; and
e. a scoring of each non-scheduled pharmaceutical by an anticipation of problems in the pharmaceutical dispensing system.

23-25. (canceled)

26. The pharmaceutical dispensing system according to claim 22, wherein said anticipation of needs are based on data from one or more of run models, real-time data, historical data, physical location of users expected to need the pharmaceuticals, expected location of users at an expected time of dispensing.

27. (canceled)

28. The pharmaceutical dispensing system according to claim 22, wherein said anticipation of problems are based on data from one or more of scheduled maintenance, run models and historical data.

29. The pharmaceutical dispensing system according to claim 18, wherein said at least one first pharmaceutical dispensing machine is further configured for storing, packing and dispensing non-scheduled pharmaceuticals in response to at least one non-scheduled demand.

30. The pharmaceutical dispensing system according to claim 18, wherein said at least one second pharmaceutical dispensing machine is further configured for storing, packing and dispensing pharmaceuticals in batches according to a pharmaceutical dispensing schedule.

31. The pharmaceutical dispensing system according to claim 18, wherein said dispense non-scheduled pharmaceuticals in response to at least one non-scheduled demand is done immediately after the at least one non-scheduled demand is received.

32. The pharmaceutical dispensing system according to claim 22, wherein said expected disruption caused to said schedule is caused by disruption of a pharmaceutical dispensing process of said pharmaceuticals in batches; and
wherein said expected disruption of said pharmaceutical dispensing process of said pharmaceuticals in batches is caused by said dispensing of said non-scheduled pharmaceuticals in response to said at least one non-scheduled demand.

33-40. (canceled)
